(19) United States
(12) Patent Application Publication
(10) Pub. No.: US 2023/0360734 A1
(43) Pub. Date: Nov. 9, 2023

(54) TRAINING PROTEIN STRUCTURE PREDICTION NEURAL NETWORKS USING REDUCED MULTIPLE SEQUENCE ALIGNMENTS

(71) Applicant: DeepMind Technologies Limited, London (GB)

(72) Inventors: Richard Andrew Evans, London (GB); John Jumper, London (GB); Timothy Frederick Goldie Green, London (GB); David Reiman, Santa Cruz, CA (US)

(21) Appl. No.: 18/025,689

(22) PCT Filed: Aug. 12, 2021
(86) PCT No.: PCT/EP2021/072552

§ 371 Date: Mar. 10, 2023

Related U.S. Application Data
(60) Provisional application No. 63/107,362, filed on Oct. 29, 2020.

(51) Int. Cl. G16B 40/20; G16B 15/20; G16B 30/10; G06N 3/045

(52) U.S. Cl. CPC G16B 40/20; G16B 15/20; G16B 30/10; G06N 3/045

(57) ABSTRACT

Methods, systems, and apparatus, including computer programs encoded on a computer storage medium, for training neural networks to predict the structure of a protein. In one aspect, a method comprises: obtaining, for each of a plurality of proteins, a full multiple sequence alignment for the protein; generating, for each of the plurality of proteins, target structure parameters characterizing a structure of the protein from the full multiple sequence alignment for the protein, comprising processing a representation of the full multiple sequence alignment for the protein using the structure prediction neural network to generate output structure parameters characterizing a structure of the protein, and determining the target structure parameters for the protein based on the output structure parameters for the protein; determining, for each of the plurality of proteins, a reduced multiple sequence alignment for the protein, comprising removing or masking data from the full multiple sequence alignment for the protein.

### BACKGROUND

This specification relates to training neural networks that predict protein structure.

A protein is specified by one or more sequences of amino acids. An amino acid is an organic compound which includes an amino functional group and a carboxyl functional group, as well as a side-chain (i.e., group of atoms) that is specific to the amino acid. Protein folding refers to a physical process by which a sequence of amino acids folds into a three-dimensional (3-D) configuration. The structure of a protein defines the 3-D configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes training systems implemented as computer programs on one or more computers in one or more locations for training structure prediction neural networks that can predict protein structures.

As used throughout this specification, the term “protein” can be understood to refer to any biological molecule that is specified by one or more sequences of amino acids. For example, the term protein may be understood to refer to a protein domain (e.g., a portion of an amino acid sequence that can undergo protein folding nearly independently of the rest of the amino acid sequence) or a protein complex (e.g., that is specified by multiple associated amino acid sequences).

The methods and systems described herein can be used to train a structure prediction neural network to be used to obtain a ligand such as a drug or a ligand of an industrial enzyme. For example, a method of obtaining a ligand may include obtaining a target amino acid sequence, in particular the amino acid sequence of a target protein, and processing an input based on the target amino acid sequence using the structure prediction neural network to determine a (tertiary) structure of the target protein, i.e., the predicted protein structure. The method may then include evaluating an interaction of one or more candidate ligands with the structure of the target protein. The method may further include selecting one or more of the candidate ligands as the ligand dependent on a result of the evaluating of the interaction.

In some implementations, evaluating the interaction may include evaluating binding of the candidate ligand with the structure of the target protein. For example, evaluating the interaction may include identifying a ligand that binds with sufficient affinity for a biological effect. In some other implementations, evaluating the interaction may include evaluating an association of the candidate ligand with the structure of the target protein which has an effect on a function of the target protein, e.g., an enzyme. The evaluating may include evaluating an affinity between the candidate ligand and the structure of the target protein, or evaluating a selectivity of the interaction.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand.

The evaluation of the interaction of a candidate ligand with the structure of the target protein may be performed using a computer-aided approach in which graphical models of the candidate ligand and target protein structure are displayed for user-manipulation, and/or the evaluation may be performed partially or completely automatically, for example using standard molecular (protein-ligand) docking software. In some implementations the evaluation may include determining an interaction score for the candidate ligand, where the interaction score includes a measure of an interaction between the candidate ligand and the target protein. The interaction score may be dependent upon a strength and/or specificity of the interaction, e.g., a score dependent on binding free energy. A candidate ligand may be selected dependent upon its score.

In some implementations the target protein includes a receptor or enzyme and the ligand is an agonist or antagonist of the receptor or enzyme. In some implementations the method may be used to identify the structure of a cell surface marker. This may then be used to identify a ligand, e.g., an antibody or a label such as a fluorescent label, which binds to the cell surface marker. This may be used to identify and/or treat cancerous cells.

In some implementations the candidate ligand(s) may include small molecule ligands, e.g., organic compounds with a molecular weight of <900 daltons. In some other implementations the candidate ligand(s) may include polypeptide ligands, i.e., defined by an amino acid sequence.

In some cases, a structure prediction neural network that is trained using the techniques described herein can be used to determine the structure of a candidate polypeptide ligand, e.g., a drug or a ligand of an industrial enzyme. The interaction of this with a target protein structure may then be evaluated; the target protein structure may have been determined using a structure prediction neural network or using conventional physical investigation techniques such as x-ray crystallography and/or magnetic resonance techniques.

Thus in another aspect there is provided a method of using a structure prediction neural network that is trained using the techniques described herein to obtain a polypeptide ligand (e.g., the molecule or its sequence). The method may include obtaining an amino acid sequence of one or more candidate polypeptide ligands. The method may further include using the structure prediction neural network to determine (tertiary) structures of the candidate polypeptide ligands. The method may further include obtaining a target protein structure of a target protein, in silico and/or by physical investigation, and evaluating an interaction between the structure of each of the one or more candidate polypeptide ligands and the target protein structure. The method may further include selecting one or more of the candidate polypeptide ligands as the polypeptide ligand dependent on a result of the evaluation.

As before evaluating the interaction may include evaluating binding of the candidate polypeptide ligand with the structure of the target protein, e.g., identifying a ligand that binds with sufficient affinity for a biological effect, and/or evaluating an association of the candidate polypeptide ligand with the structure of the target protein which has an effect on a function of the target protein, e.g., an enzyme, and/or evaluating an affinity between the candidate polypeptide ligand and the structure of the target protein, or evaluating a selectivity of the interaction. In some implementations the polypeptide ligand may be an aptamer.

Implementations of the method may further include synthesizing, i.e., making, the small molecule or polypeptide ligand. The ligand may be synthesized by any conventional chemical techniques and/or may already be available, e.g., may be from a compound library or may have been synthesized using combinatorial chemistry. The synthesis may be manual, or semi- or wholly automatic. The synthesized small molecule or polypeptide ligand may be a drug.

The method may further include testing the ligand for biological activity in vitro and/or in vivo. For example the ligand may be tested for ADME (absorption, distribution, metabolism, excretion) and/or toxicological properties, to screen out unsuitable ligands. The testing may include, e.g., bringing the candidate small molecule or polypeptide ligand into contact with the target protein and measuring a change in expression or activity of the protein.

In some implementations a candidate (polypeptide) ligand may include: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region. Thus in some implementations the method is used to obtain a polypeptide ligand comprising an antibody.

Misfolded proteins are associated with a number of diseases. Thus in a further aspect there is provided a method of using a structure prediction neural network that is trained using the techniques described herein to identify the presence of a protein mis-folding disease. The method may include obtaining an amino acid sequence of a protein and using the structure prediction neural network to determine a structure of the protein. The method may further include obtaining a structure of a version of the protein obtained from a human or animal body, e.g., by conventional (physical) methods, such as X-ray crystallography, NMR spectroscopy or electron microscopy. The method may then include comparing the structure of the protein with the structure of the version obtained from the body and identifying the presence of a protein mis-folding disease dependent upon a result of the comparison. That is, mis-folding of the version of the protein from the body may be determined by comparison with the in silico determined structure.

In some other aspects a computer-implemented method as described above or herein may be used to identify active/binding/blocking sites on a target protein from its amino acid sequence.

According to another aspect there is provided a system comprising: one or more computers; and one or more storage devices communicatively coupled to the one or more computers, wherein the one or more storage devices store instructions that, when executed by the one or more computers, cause the one or more computers to perform operations to implement the techniques described herein. The system may include a subsystem, e.g. a robotic protein synthesis subsystem, to make a protein obtained using the techniques.

Particular embodiments of the subject matter described in this specification can be implemented so as to realize one or more of the following advantages.

This specification describes a training system that can train a structure prediction neural network using both “paired” and “unpaired” training examples. Each paired training example includes a multiple sequence alignment (MSA) for a protein and the ground truth (e.g., actual) protein structure, and the training system can train the structure prediction neural network to process the MSA to generate a predicted protein structure that matches the ground truth protein structure. Each unpaired training example includes a MSA for a protein, but the ground truth structure of the protein may be unknown. To train the structure prediction neural network on the unpaired training examples, the training system generates a prediction target for each unpaired training example by processing the MSA from the unpaired training example using the structure prediction neural network to generate a target protein structure. The training system then trains the structure prediction neural network to, for each unpaired training example, process a “reduced” MSA, i.e., where some of the data in the MSA has been removed or masked, to generate a predicted protein structure that matches the corresponding target protein structure.

By training the structure prediction neural network using unpaired training examples, the training system can improve the performance (e.g., prediction accuracy) of the structure prediction neural network by reducing the likelihood of the structure prediction neural network overfitting the paired training examples. The structure prediction neural network can “overfit” the paired training examples, e.g., by learning to predict the ground truth protein structures specified by the paired training examples based on irrelevant variations in the MSAs, rather than based on implicit reasoning rooted in inferred bio-chemical principles. Moreover, the number of available unpaired training examples may be far greater than the number of available paired training examples, and therefore training the structure prediction neural network on the unpaired training examples can enable it to learn to effectively predict structures of a wider variety of proteins.

This specification describes a training system for training a “student” structure prediction neural network that can predict the structure of a protein by processing an input that includes a representation of the amino acid sequence of the protein, but does not include a MSA for the protein. To increase the amount of training data available beyond only paired training examples (i.e., where the ground truth protein structure is known), the training system trains a “teacher” structure prediction neural network that can accurately predict the structure of a protein by processing an input that includes a MSA for the protein. The training system uses the teacher structure prediction neural network to generate a prediction target for each unpaired training example by processing an input including the MSA from the unpaired training example to generate a target protein structure. The training system then trains the student structure prediction neural network to, for each unpaired training example, generate a predicted protein structure that matches the target protein structure for the training example without processing the protein MSA.

By using the teacher structure prediction neural network to generate prediction targets, the training system can substantially increase the amount of training data available for training the student structure prediction neural network and thereby enable the student structure prediction neural network to be trained to achieve higher prediction accuracy. After training, the student structure prediction neural network can be used to predict the structure of any protein, regardless of whether a MSA for the protein is available, thereby making the student structure prediction neural network broadly applicable to any task that requires predicting protein structures.

Identifying ground truth protein structures can be expensive and time consuming, and the ground truth structures for many proteins may not be known. The training systems described in this specification enable a structure prediction neural network to be trained to effectively predict structures of a wide variety of proteins, even in the absence of ground truth structures for many proteins.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A-B describe a training system for training a structure prediction neural network that can predict the structure of a protein by processing an input that includes a MSA for the protein.

FIG. 2 describes a training system for training a structure prediction neural network that can predict the structure of a protein without processing a MSA for the protein.

FIG. 3 is an illustration of an unfolded protein and a folded protein.

FIG. 4 is a flow diagram of an example process for training a structure prediction neural network that is configured to generate structure parameters that characterize a structure of a protein by processing a network input that comprises a representation of a multiple sequence alignment for the protein.

FIG. 5 is a flow diagram of an example process for training a structure prediction neural network that can generate structure parameters characterizing a structure of a protein without processing a multiple sequence alignment for the protein.

Like reference numbers and designations in the various drawings indicate like elements.

### DETAILED DESCRIPTION

This specification describes training systems that can train a protein structure prediction neural network having a set of model parameters, e.g., by repeatedly adjusting the current values of the model parameters to determine trained values of the model parameters from initial values of the model parameters.

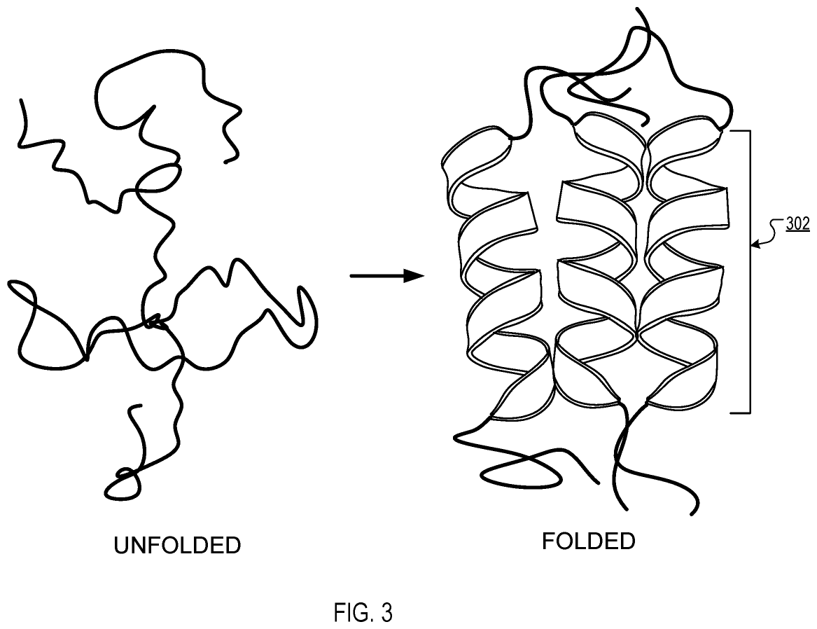

Throughout this specification, a protein structure prediction neural network (or “structure prediction neural network”) refers to a neural network that processes an input characterizing a protein to generate an output that includes a set of structure parameters that characterize a predicted structure of the protein. The structure of a protein refers to the three-dimensional (3-D) configuration of the atoms in the protein after the protein undergoes protein folding. FIG. 3 provides an illustration of an unfolded protein and a folded protein.

For convenience, this specification will refer primarily to training neural networks to perform protein structure prediction. However, the techniques described herein are broadly applicable to training any machine learning model (i.e., having a set of trainable model parameters) to perform protein structure prediction. Other examples of machine learning models can include, e.g., random forest models and support vector machine models.

To generate structure parameters that characterize a predicted structure of a protein, a structure prediction neural network can process an input that includes a representation of the amino acid sequence of the protein, and in some cases, a representation of a multiple sequence alignment (MSA) for the protein. The MSA can specify a sequence alignment of the amino acid sequence of the protein with multiple additional amino acid sequences, e.g., from other proteins, e.g., homologous proteins. More specifically, the MSA can define a correspondence between the positions in the amino acid sequence of the protein and corresponding positions in the amino acid sequences of multiple additional proteins. The MSA can be generated, e.g., by processing a database of amino acid sequences using any appropriate computational sequence alignment technique, e.g., progressive alignment construction. The amino acid sequences in the MSA can be understood as having an evolutionary relationship, e.g., where each amino acid sequence in the MSA may share a common ancestor. The correlations between the amino acid sequences in the MSA can encode information that is relevant to predicting the structure of the protein. The MSA can be obtained by any known technique, such as those reviewed as https://en.wikipedia.org/wiki/Multiple_sequence_alignment.

A representation of an amino acid sequence of a protein may be an ordered collection of embeddings that includes a respective embedding (i.e., an ordered collection of numerical values, e.g., a vector or matrix of numerical values) corresponding to each position in the amino acid sequence. The respective embedding corresponding to each position in the amino acid sequence may be, e.g., a one-hot vector that defines the identity of the amino acid at the position in the amino acid sequence. A one-hot vector has a different component corresponding to each possible amino acid (e.g., of a predetermined number of possible amino acids). A one-hot vector representing a particular amino acid has value one (or some other predetermined value) in the component corresponding to the particular amino acid and value zero (or some other predetermined value) in the other components.

A representation of a MSA for a protein may be an ordered collection of embeddings that includes a respective embedding corresponding to each position in each amino acid sequence in the MSA. The respective embedding corresponding to each position in each amino acid sequence may be, e.g., a one-hot vector that defines the identity of the amino acid in the position of the amino acid sequence. In some cases, a representation of a MSA for a protein may be a set of features derived from the MSA, e.g., second order statistical features such as those described with reference to: S. Seemayer, M. Gruber, and J. Soding: “CCMpred: fast and precise prediction of protein residue-residue contacts from correlated mutations”, Bioinformatics, 2014.

In some implementations, the structure parameters generated by a structure prediction neural network for a protein can include a sequence of three-dimensional (3D) numerical coordinates, where each coordinate represents the spatial position (in some given frame of reference) of a corresponding atom in an amino acid of the protein. In a particular example, the structure parameters may comprise a sequence of 3D numerical coordinates representing the respective spatial positions of the alpha carbon atoms in the amino acids in the protein. An alpha carbon atom, which may be referred to in this specification as a backbone atom, refers to a carbon atom in an amino acid to which the amino functional group, the carboxyl functional group, and the side-chain are bonded. Alternatively or additionally, the structure parameters may comprise a sequence of torsion (i.e., dihedral) angles between specific atoms in the amino acids of the protein. For example, the structure parameters may be a sequence of phi (ϕ), psi (ψ), and omega (ω) dihedral angles between the backbone atoms in the amino acids of the protein.

In some implementations, the structure parameters generated by a structure prediction neural network for a protein can include a “distance map” that characterizes a respective estimated distance (e.g., measured in angstroms) between each pair of amino acids in the protein. In some examples, a distance map can characterize the estimated distance between a pair of amino acids by a probability distribution over a set of possible distances between the pair of amino acids. A distance map may be represented as an ordered collection of numerical values, e.g., a vector or matrix of numerical values.

Generally, the structure prediction neural networks described in this specification can have any appropriate neural network architectures that enable them to perform their described functions. For example, the structure prediction neural networks can have respective architectures that include any appropriate types of neural network layers (e.g., fully-connected layers, convolutional layers, pooling layers, self-attention layers, etc.), arranged in any appropriate configuration (e.g., as a linear sequence of layers).

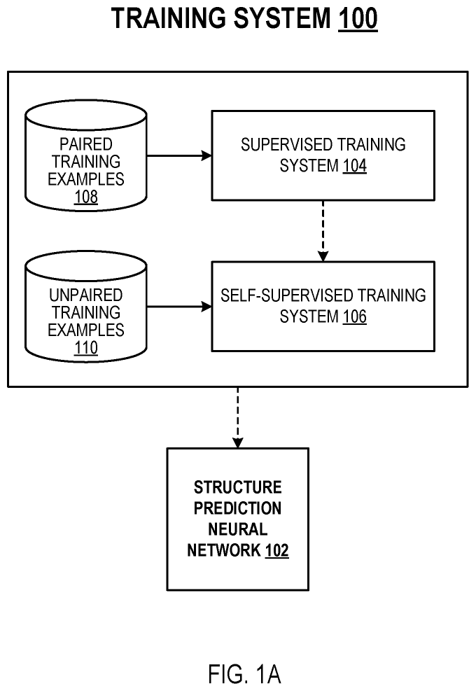

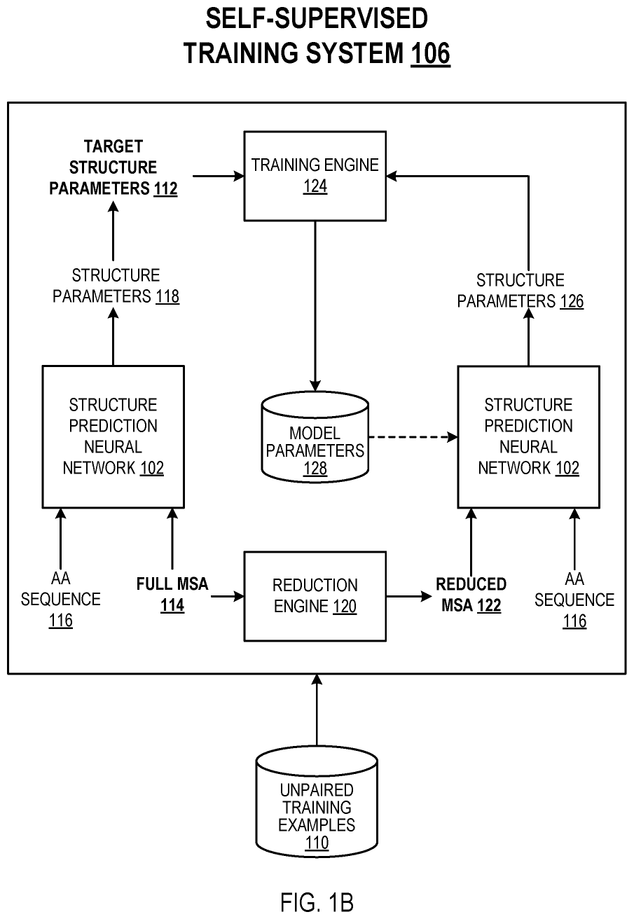

FIG. 1A-B, which will be described in more detail below, describe a training system for training a structure prediction neural network that can predict the structure of a protein by processing an input that includes a MSA for the protein.

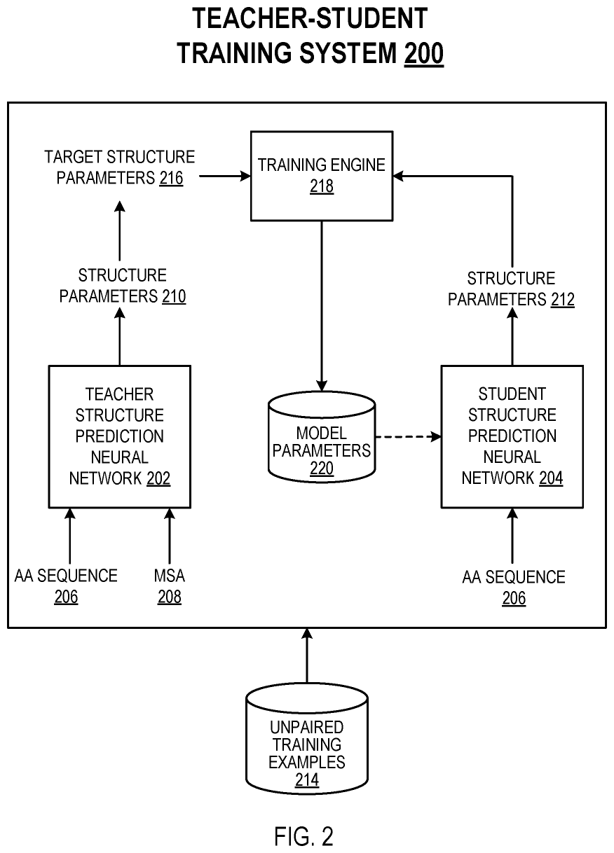

FIG. 2, which will be described in more detail below, describes a training system for training a neural network that can predict the structure of a protein without processing a MSA for the protein.

FIG. 1A shows an example training system 100. The training system 100 is an example of a system implemented as computer programs on one or more computers in one or more locations in which the systems, components, and techniques described below are implemented.

The training system 100 is configured to train a structure prediction neural network 102 that can generate structure parameters that characterize a structure of a protein by processing an input that includes respective representations of: (i) the amino acid sequence of the protein, and (ii) a MSA for the protein.

The training system 100 trains the structure prediction neural network 102 using a supervised training system 104 and a self-supervised training system 106.

The supervised training system 104 trains the structure prediction neural network 102 on a set of “paired” training examples 108. Each paired training example 108 corresponds to a respective protein and includes data defining: (i) a training input to the structure prediction neural network that includes the amino acid sequence of the protein and a MSA for the protein, and (ii) a ground truth structure of the protein. The ground truth structure of the protein refers to a known structure of the protein that may have been determined experimentally using physical (i.e. real-world) instances of the protein by physical laboratory techniques (e.g., x-ray crystallography) or by some other technique. The ground truth structure of the protein may be in the form of respective values for a plurality of ground truth structure parameters. The ground truth structure parameters may be respectively the structure parameters generated by the structure prediction neural network.

The supervised training system 104 can train the structure prediction neural network to generate structure parameters that match the ground truth structure parameters specified by the paired training examples 108. More specifically, the supervised training system 104 can train the structure prediction neural network 102 to optimize an objective function that measures an error between: (i) the structure parameters generated by the structure prediction neural network, and (ii) the ground truth structure parameters specified by the paired training examples. The objective function can measure the error between respective sets of structure parameters, e.g., as a squared-error, or in any other appropriate manner. The supervised training system 104 can train the structure prediction neural network 102 using any appropriate training technique, e.g., stochastic gradient descent.

Optionally, the supervised training system 104 can train the structure prediction neural network 102 to generate one or more auxiliary outputs. Training the structure prediction neural network to generate auxiliary outputs can allow the structure prediction neural network to be trained more rapidly and to achieve a higher prediction accuracy, e.g., by enabling the structure prediction neural network to generate more effective internal representations of proteins. A few examples of auxiliary outputs are described next.

In one example, the supervised training system 104 can train the structure prediction neural network to process an input characterizing a protein to generate an auxiliary output that estimates a confidence in the accuracy of the structure parameters generated by the structure prediction neural network for the protein. More specifically, the auxiliary output can estimate an error (e.g., a squared-error) between: (i) the structure parameters generated by the structure prediction neural network for the protein, and (ii) the ground truth structure parameters for the protein.

In another example, the supervised training system 104 can mask the identities of respective amino acids at one or more positions in one or more amino acid sequences in the MSA provided as an input to the structure prediction neural network 102. In this example, the supervised training system 104 can train the structure prediction neural network 102 to generate an auxiliary output that predicts the identity of each masked amino acid in the input MSA. “Masking” the identity of an amino acid at a position in a MSA can refer to replacing the data identifying the amino acid at the position by a predefined masking identifier (token). The supervised training system can randomly select the positions of the amino acids to be masked in the MSA.

The self-supervised training system 106 trains the structure prediction neural network on a set of “unpaired” training examples 110. Each unpaired training example 110 corresponds to a respective protein and includes data defining a training input to the structure prediction neural network that includes the amino acid sequence of the protein and a MSA for the protein. In contrast to the paired training examples 108, the ground truth protein structure may be unknown for some or all of the unpaired training examples.

To train the structure prediction neural network 102, the self-supervised training system 106 can process the MSA included in each unpaired training example to generate a “reduced” MSA, e.g., by randomly removing or masking data from the full MSA (i.e. the whole of the MSA in the training example 110, which typically includes respective data for substantially every amino acid in the respective protein). The self-supervised training system 106 can generate data defining respective “target” structure parameters for each unpaired training example 110 based on a set of structure parameters generated by the structure prediction neural network 102 by processing an input that includes the full (i.e., unreduced) MSA from the unpaired training example. The self-supervised training system 106 can then train the structure prediction neural network to process the reduced MSA for each unpaired training example to generate structure parameters that match the target structure parameters for the training example. An example of a self-supervised training system 106 is described in more detail with reference to FIG. 1B.

The training system 100 trains the structure prediction neural network 102 using both the supervised training system 104 and the self-supervised training system 106. For example, the training system 100 may first train the structure prediction neural network 102 using the supervised training system 104, and then using the self-supervised training system 106. In some implementations, the training system 100 may repeatedly alternate between training the structure prediction neural network 102 using the supervised training system 104 and the self-supervised training system 106.

Training the structure prediction neural network 102 using the self-supervised training system 106 can improve the performance (e.g., prediction accuracy) of the structure prediction neural network 102 by reducing the likelihood of the structure prediction neural network overfitting the paired training examples 108. The structure prediction neural network 102 can “overfit” the paired training examples, e.g., by learning to predict the ground truth protein structures specified by the paired training examples based on irrelevant variations in the training inputs, rather than based on implicit reasoning rooted in inferred bio-chemical principles. Moreover, the number of available unpaired training examples may be far greater than the number of available paired training examples, and therefore the self-supervised training system 106 can enable the structure prediction neural network 102 to learn to effectively predict structures of a wider variety of proteins.

FIG. 1B shows an example self-supervised training system 106. The self-supervised training system 106 is an example of a system implemented as computer programs on one or more computers in one or more locations in which the systems, components, and techniques described below are implemented.

The self-supervised training system 106 trains the structure prediction neural network 102 on a set of unpaired training examples 110. Each unpaired training example 110 corresponds to a respective protein and includes data defining a training input to the structure prediction neural network 102 that includes: (i) the amino acid sequence of the protein, and (ii) a “full” (i.e., unreduced) MSA for the protein. Generally, the ground truth protein structure may be unknown for some or all of the unpaired training examples.

As part of training the structure prediction neural network 102, the self-supervised training system 106 generates a respective set of target structure parameters 112 for each unpaired training example 110. The target structure parameters 112 for an unpaired training example characterize a predicted structure of the protein corresponding to the unpaired training example. The target structure parameters 112 provide a prediction target for the structure prediction neural network 102 when processing a reduced MSA rather than the full MSA for the protein, as will be described in more detail below.

To generate the target structure parameters 112 for an unpaired training example 110, the structure prediction neural network 102 processes an input including representations of the full MSA 114 and the amino acid (AA) sequence 116 for the protein to generate output structure parameters 118. The self-supervised training system 106 then determines the target structure parameters 112 based on the structure parameters 118 generated by the structure prediction neural network 102 by processing the full MSA 114 and the AA sequence 116. In some implementations, the self-supervised training system 106 may determine the target structure parameters 112 to be equal to the structure parameters 118 generated by the structure prediction neural network. In some implementations, the self-supervised training system 106 may determine the target structure parameters 112 by adding random noise values to the structure parameters 118 generated by the structure prediction neural network 102. Adding random noise values to the structure parameters 118 generated by the structure prediction neural network 102 as part of generating the target structure parameters 112 may reduce the likelihood of overfitting and thereby regularize the training of the structure prediction neural network 102.

In addition to generating the target structure parameters 112 for each unpaired training example 110, the self-supervised training system 106 processes the full MSA 114 from each unpaired training example 110 using a reduction engine 120 to generate a corresponding “reduced” MSA 122. The reduction engine 120 can process a full MSA 114 to generate a reduced MSA 122, e.g., by randomly removing or masking data from the full MSA 114. A few examples of operations that can be performed by the reduction engine 120 to generate a reduced MSA 122 from a full MSA 114 are described in more detail next.

In some implementations, the reduction engine 120 can randomly remove one or more amino acid sequences from a full MSA 114 as part of generating a reduced MSA 122. The reduction engine 120 can determine how many amino acid sequences to remove from the full MSA 114, and which particular amino acid sequences to remove from the full MSA 114, using a stochastic procedure. For example, the reduction engine 120 may sample a reduction parameter value, in accordance with a probability distribution over a space of possible reduction parameter values, that defines a number of amino acid sequences to be removed from the full MSA 114. The space of possible reduction parameter values can be, e.g., the interval (0,1), and the sampled reduction parameter value can define the fraction of amino acid sequences to be removed from the full MSA 114. For example, sampling a reduction parameter value of 0.15 may define that 15% of the amino acid sequences in the full MSA 114 should be removed. After sampling the reduction parameter value, the reduction engine 120 can randomly remove the specified number of amino acid sequences from the full MSA 114.

In some implementations, the reduction engine 120 can randomly mask the identity of the respective amino acid at one or more positions in one or more amino acid sequences in the full MSA 114. “Masking” the identity of an amino acid at a position in the full MSA 114 can refer to replacing the data identifying the amino acid at the position by a predefined masking identifier (token). In one example, the reduction engine 120 may sample a masking parameter value in accordance with a probability distribution over a space of possible masking parameter values, e.g., the interval (0, 0.05). The masking parameter value can define the probability that the identity of the respective amino acid any position in any of the amino acid sequences of the full MSA should be masked. After sampling the masking parameter value, the reduction engine 120 can mask the identity of each amino acid in each amino acid sequence in the MSA with the probability defined by the masking parameter value.

The self-supervised training system 106 trains the structure prediction neural network 102 to, for each unpaired training example, process representations of: (i) the AA sequence 116, and (ii) the reduced MSA 122, to generate structure parameters 126 that match the target structure parameters 112 for the unpaired training example. More specifically, the self-supervised training system 106 uses a training engine 124 to train the structure prediction neural network 102 to optimize an objective function. The objective function can measure an error between, for each unpaired training example: (i) the structure parameters 126 generated by the structure prediction neural network from the reduced MSA 122, and (ii) the target structure parameters 112 generated from the full MSA 114. The objective function can measure the error between respective sets of structure parameters, e.g., as a squared-error, or in any other appropriate manner.

The self-supervised training system 106 can use a training engine 124 to train the structure prediction neural network 102 using any appropriate training technique, e.g., by stochastic gradient descent over a sequence of training iterations. More specifically, at each training iteration, the training engine 124 can sample a batch of unpaired training examples. For each unpaired training example in the batch, the structure prediction neural network 102 can process the corresponding reduced MSA 122 and AA sequence 116 in accordance with the current values of the model parameters 128 of the structure prediction neural network 102 to generate corresponding structure parameters 126. The training engine 124 can then evaluate an objective function that measures the error between: (i) the target structure parameters 112, and (ii) the structure parameters 126 generated by the structure prediction neural network 102 for the unpaired training examples in the current batch. The training engine 124 can determine gradients of the objective function with respect to the model parameters of the structure prediction neural network, e.g., by backpropagation, and use the gradients to update the current values of the model parameters using any appropriate gradient descent optimization technique, e.g., RMSprop or Adam.

In some implementations, the self-supervised training system 106 can train the structure prediction neural network 102 to generate one or more auxiliary outputs, e.g., an auxiliary output that predicts the identity of each masked amino acid in the reduced MSA 122.

The structure parameters 118 generated by the structure prediction neural network based on a full MSA 114 may be inaccurate for one or more of the unpaired training examples. As a result, the target structure parameters 112 for these training examples can be inaccurate, and using these target structure parameters 112 during training may decrease the performance of the structure prediction neural network 102, e.g., by reinforcing errors made by the structure prediction neural network 102.

To reduce the likelihood of inaccurate target structure parameters 112 negatively affecting the training of the structure prediction neural network 102, the self-supervised training system 106 may estimate a respective confidence in the target structure parameters 112 for each unpaired training example. In some implementations, the self-supervised training system 106 may refrain from training the structure prediction neural network on any target structure parameters 112 for which the confidence estimate does not satisfy a threshold. In some implementations, the self-supervised training system 106 may condition the objective function on the confidence estimates in the target structure parameters 112 for each training example, e.g., to reduce the influence of low-confidence target structure parameters 112 on the objective function. For example, the objective function [symbol] may be given by:

$$ℒ = {\sum }_{i = 1}^{N} {c}_{i} \cdot Err ( {T}_{i} , {P}_{i} ) \qquad ( 1 )$$

where i indexes the N training examples, cᵢ denotes a scaling factor based on a confidence in the target structure parameters for training example i, Tᵢ denotes the target structure parameters for training example i, Pᵢ denotes the structure parameters generated by the structure prediction neural network based on the reduced MSA for training example i, and Err (.,.) denotes an error measure, e.g., a squared-error.

The self-supervised training system 106 may determine confidence estimates for the target structure parameters 112 in a variety of ways. A few example ways to determine a confidence estimate for the target structure parameters 112 for a training example are described in more detail next.

In one example, the self-supervised training system 106 may obtain a confidence estimate for the target structure parameters 112 for a training example as an auxiliary output that is generated by the structure prediction neural network 102 by processing the full MSA 114 for the training example. Generating a confidence estimate as an auxiliary output of the structure prediction neural network 102 is described in more detail with reference to FIG. 1A.

In another example, the self-supervised training system 106 may obtain a confidence estimate for the target structure parameters 112 for a training example based on an estimated distance map for the protein corresponding to the training example. The distance map can define, for each pair of amino acids in the protein, a probability distribution over a range of possible physical distances between the pair of amino acids in the protein structure. The self-supervised training system 106 may obtain the distance map as an auxiliary or main output that is generated by the structure prediction neural network by processing the full MSA 114 for the training example. The self-supervised training system 106 can determine the confidence estimate based on, for each pair of amino acids, a difference between: (i) the probability distribution defined by the distance map over possible distances between the pair of amino acids, and (ii) a “background” probability distribution.

The background probability distribution may be a predefined probability distribution over a range of possible distances that reflects the statistical distribution of distances between pairs of amino acids in known protein structures. A difference between respective probability distributions may be determined, e.g., as a Kullback-Leibler divergence. Generally, greater differences between the probability distributions defined by the distance map and the background probability distribution can indicate a higher confidence in the target structure parameters generated by the structure prediction neural network for the training example.

After training the structure prediction neural network 102 to process the reduced MSA 122 to generate the corresponding target structure parameters 112 for each training example, the self-supervised training system 106 may provide the trained model parameters 128 of the structure prediction neural network 102 as an output.

Optionally, the self-supervised training system 106 can generate new target structure parameters 112 for the training examples 110 in accordance with the trained values of the model parameters 128 of the structure prediction neural network 102, and repeat the above-described procedure to continue training the structure prediction neural network 102. In some cases, the self-supervised training system 106 can continue iteratively repeating the self-supervised training procedure for training the structure prediction neural network 102 until a termination criterion is satisfied.

In some implementations, the self-supervised training system 106 increases the expected amount of data that is removed or masked from the full MSAs 114 by the reduction engine 120 at each iteration of the training procedure. For example, the self-supervised training system 106 may, at each iteration of the training procedure, increase the mean of a probability distribution over possible reduction parameter values from which the reduction engine samples reduction parameter values that define the fraction of amino acid sequences to be removed from full MSAs. Increasing the expected amount of data that is removed or masked from the full MSAs at each iteration of the training procedure can improve the performance of the structure prediction neural network 102 at predicting protein structures by processing MSAs that include few amino acid sequences.

FIG. 2 shows an example teacher-student training system 200. The teacher-student training system 200 is an example of a system implemented as computer programs on one or more computers in one or more locations in which the systems, components, and techniques described below are implemented.

The training system 200 uses a “teacher” structure prediction neural network 202 to train a “student” structure prediction neural network 204, in particular, by using the teacher structure prediction neural network to generate target structure parameters to be used as prediction targets by the student structure prediction neural network.

The teacher structure prediction neural network 202 is configured to process an input that includes both: (i) a representation of an amino acid (AA) sequence 206 of a protein, and (ii) a representation of a MSA 208 for the protein. The teacher structure prediction neural network 202 processes the input to generate structure parameters 210 that characterize a predicted structure of the protein.

The student structure prediction neural network 204 is configured to process an input that includes a representation of an AA sequence of a protein, but does not include a representation of a MSA 208 for the protein. In some implementations, the student structure prediction neural network 204 processes an input that includes only the representation of the AA sequence of the protein. The student structure prediction neural network 204 processes the input to generate structure parameters 212 that characterize a predicted structure of the protein (in particular, without processing a representation of a MSA for the protein, in contrast to the teacher structure prediction neural network 202, which does process an input including a representation of MSA).

The teacher structure prediction neural network 202 can be trained using any appropriate machine learning training techniques. For example, the teacher structure prediction neural network 202 can be trained using the supervised training system 104 described with reference to FIG. 1A, or a combination of the supervised training system 104 and the self-supervised training system 106, as described with reference to FIG. 1A-B.

The training system 200 trains the student structure prediction neural network based on a set of unpaired training examples 214. Each unpaired training example 214 corresponds to a respective protein and includes data defining: (i) the amino acid sequence of the protein, and (ii) a multiple sequence alignment for the protein.

Generally, the ground truth protein structure may be unknown for some or all of the unpaired training examples 214. Therefore, the training system 200 uses the teacher structure prediction neural network 202 to generate a set of target structure parameters 216 for each unpaired training example 214 that characterize a predicted structure of the corresponding protein.

To generate the target structure parameters 216 for a training example 214, the training system 200 uses the teacher structure prediction neural network 202 to generate a set of structure parameters 210 for each training example 214. The teacher structure prediction neural network 202 generates the structure parameters 210 for each training example by processing an input including respective representations of: (i) the AA sequence 206 from the training example, and (ii) the MSA 208 from the training example.

The training system 200 determines the target structure parameters 216 for each training example based on the structure parameters 210 generated by the teacher structure prediction neural network 202 for the training example. In some implementations, the training system 200 may determine the target structure parameters 216 to be equal to the structure parameters 210 generated by the teacher structure prediction neural network 202. In some implementations, the training system 200 may determine the target structure parameters 216 by adding random noise values to the structure parameters 210 generated by the teacher structure prediction neural network 202. Adding random noise values to the structure parameters 210 generated by the teacher structure prediction neural network 202 as part of generating the target structure parameters 216 may reduce the likelihood of overfitting and thereby regularize the training of the student structure prediction neural network 204.

The training system 200 can train the student structure prediction neural network 204 to, for each training example, process a representation of the AA sequence 206 for the training example to generate structure parameters 212 that match the target structure parameters 216 for the training example. More specifically, the training system 200 uses a training engine 218 to train the student structure prediction neural network 204 to optimize an objective function. The objective function can measure an error between, for each training example: (i) the structure parameters 212 generated by the student structure prediction neural network by processing the AA sequence 206 for the training example, and (ii) the target structure parameters 216 for the training example. The objective function can measure the error between respective sets of structure parameters, e.g., as a squared-error, or in any other appropriate manner.

The training system 200 can train the student structure prediction neural network 204 using any appropriate training technique, e.g., by stochastic gradient descent over a sequence of training iterations. More specifically, at each training iteration, the training engine 218 can sample a batch of training examples. For each training example in the batch, the student structure prediction neural network 204 processes a representation of the corresponding AA sequence 206 in accordance with the current values of the model parameters 220 of the student structure prediction neural network 204 to generate structure parameters 212. The training engine 218 then evaluates the objective function that measures the error between: (i) the target structure parameters 216, and (ii) the structure parameters 212 generated by the student structure prediction neural network 204 for the training examples in the current batch. The training engine 218 determines gradients of the objective function with respect to the model parameters of the student structure prediction neural network and uses the gradients to update the current values of the model parameters of the student structure prediction neural network using any appropriate gradient descent optimization technique. The training engine may employ the determined gradients to improve the model parameters, e.g., by backpropagation, and the gradient descent optimization technique may be, e.g., RMSprop or Adam.

In some cases, the structure parameters 210 generated by the teacher structure prediction neural network may be inaccurate for one or more of the training examples. As a result, the target structure parameters 216 for these training examples can be inaccurate, and using these target structure parameters 216 during training may decrease the performance of the student structure prediction neural network 204.

To reduce the likelihood of inaccurate target structure parameters 216 negatively affecting the training of the student structure prediction neural network 204, the training system 200 can estimate a respective confidence in the target structure parameters 216 for each training example. In some implementations, the training system 200 may refrain from training the student structure prediction neural network on any training examples where the confidence in the target structure parameters 216 for the training example does not satisfy a threshold. In some implementations, the training system 200 may condition the objective function on the confidence in the target structure parameters 216 for each training example, e.g., to reduce the influence of low-confidence target structure parameters 216 on the objective function. For example, the objective function [symbol] may be given by:

$$ℒ = {\sum }_{i = 1}^{N} {c}_{i} \cdot Err ( {T}_{i} , {P}_{i} ) \qquad ( 2 )$$

where i indexes the N training examples, cᵢ denotes a scaling factor based on a confidence in the target structure parameters 216 for training example i, Tᵢ denotes the target structure parameters for training example i, Pᵢ denotes the structure parameters generated by the student structure prediction neural network for training example i, and Err(.,.) denotes an error measure, e.g., a squared-error.

The training system 200 may determine confidence estimates for the target structure parameters 216 in a variety of ways. A few example ways to determine a confidence estimate for the target structure parameters 216 for a training example are described in more detail next.

In one example, the training system 200 may obtain a confidence estimate for the target structure parameters 216 for a training example as an auxiliary output of the teacher structure prediction neural network 202 for the training example. Generating a confidence estimate as an auxiliary output of a structure prediction neural network is described in more detail with reference to FIG. 1A.

In another example, the training system 200 may obtain a confidence estimate for the target structure parameters 216 for a training example based on an estimated distance map for the protein corresponding to the training example. The distance map can define, for each pair of amino acids in the protein, a probability distribution over a range of possible physical distances between the pair of amino acids in the protein structure. The training system 200 may obtain the distance map as an auxiliary or main output of the teacher structure prediction neural network 202. Generating a confidence estimate for a set of structure parameters based on an estimated distance map is described in more detail above with reference to FIG. 1B.

After training the student structure prediction neural network 204, the training system 200 may provide the trained model parameters 220 of the student structure prediction neural network 204 as an output.

The student structure prediction neural network 204 can predict the structure of any protein based on the amino acid sequence of the protein, without requiring a MSA for the protein. Therefore, the student structure prediction neural network may be more broadly applicable than the teacher structure, e.g., because MSAs may be unavailable for many proteins.

FIG. 3 is an illustration of an unfolded protein and a folded protein. The unfolded protein is a random coil of amino acids. The unfolded protein undergoes protein folding and folds into a 3D configuration. Protein structures often include stable local folding patterns such alpha helices (e.g., as depicted by 302) and beta sheets.

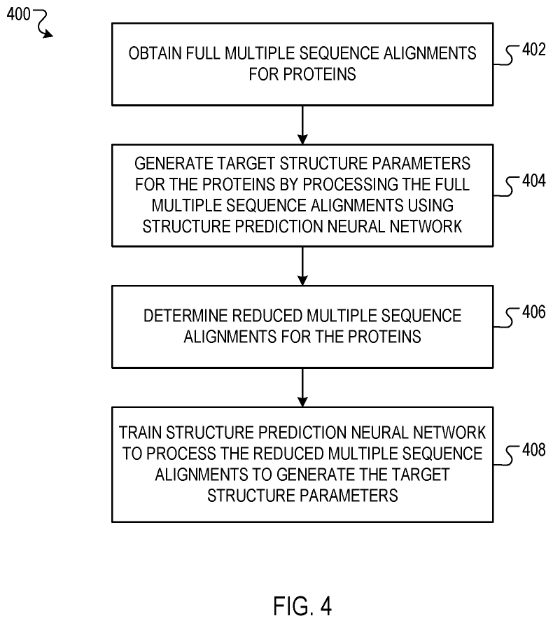

FIG. 4 is a flow diagram of an example process 400 for training a structure prediction neural network that is configured to generate structure parameters that characterize a structure of a protein by processing a network input that comprises a representation of a multiple sequence alignment for the protein. For convenience, the process 400 will be described as being performed by a system of one or more computers located in one or more locations. For example, a training system, e.g., the training system 100 of FIG. 1A, appropriately programmed in accordance with this specification, can perform the process 400.

The system obtains, for each of multiple proteins, a full multiple sequence alignment for the protein (402).

The system generates, for each of the proteins, target structure parameters characterizing a structure of the protein from the full multiple sequence alignment for the protein (404). More specifically, the system processes a representation of the full multiple sequence alignment for each protein using the structure prediction neural network to generate output structure parameters, and determines the target structure parameters for the protein based on the output structure parameters for the protein.

The system determines, for each protein, a reduced multiple sequence alignment for the protein, e.g., by removing or masking data from the full multiple sequence alignment for the protein (406).

The system trains the structure prediction neural network to, for one or more of the proteins, process a representation of the reduced multiple sequence alignment for the protein to generate structure parameters that match the target structure parameters for the protein (408).

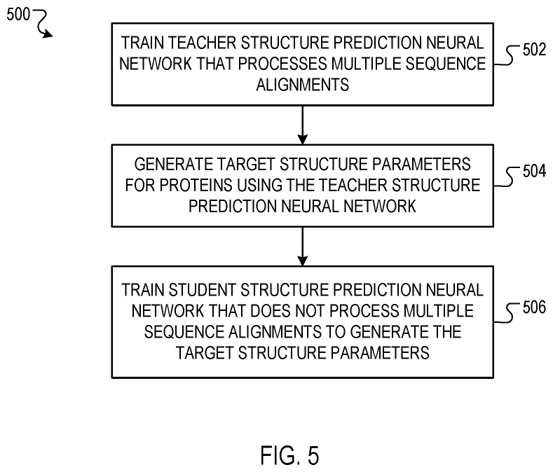

FIG. 5 is a flow diagram of an example process 500 for training a structure prediction neural network that can generate structure parameters characterizing a structure of a protein without processing a multiple sequence alignment for the protein. For convenience, the process 500 will be described as being performed by a system of one or more computers located in one or more locations. For example, a teacher-student training system, e.g., the teacher-student training system 200 of FIG. 2, appropriately programmed in accordance with this specification, can perform the process 500.

The system trains a teacher structure prediction neural network that is configured to generate structure parameters characterizing a structure of a protein by processing an input that includes respective representations of: (i) an amino acid sequence of the protein, and (ii) a multiple sequence alignment for the protein (502).

The system generates respective target structure parameters for each of multiple proteins using the teacher structure prediction neural network (504).

The system trains a student structure prediction neural network that is configured to generate structure parameters characterizing a structure of a protein by processing an input that: (i) includes a representation of an amino acid sequence of the protein, and (ii) does not include a representation of a multiple sequence alignment for the protein (506). The system trains the student structure prediction neural network to, for each protein, generate structure parameters characterizing a structure of the protein that match the target structure parameters for the protein.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus for training a structure prediction neural network that is configured to generate structure parameters that characterize a structure of a protein by processing a network input that comprises a representation of a multiple sequence alignment for the protein, the method comprising:
obtaining, for each of a plurality of proteins, a full multiple sequence alignment for the protein;
generating, for each of the plurality of proteins, target structure parameters characterizing a structure of the protein from the full multiple sequence alignment for the protein, comprising:
processing a representation of the full multiple sequence alignment for the protein using the structure prediction neural network to generate output structure parameters characterizing a structure of the protein; and
determining the target structure parameters for the protein based on the output structure parameters for the protein;
determining, for each of the plurality of proteins, a reduced multiple sequence alignment for the protein, comprising removing or masking data from the full multiple sequence alignment for the protein; and
training the structure prediction neural network to, for one or more of the plurality of proteins, process a representation of the reduced multiple sequence alignment for the protein to generate structure parameters that match the target structure parameters for the protein.

2. The method of claim 1, wherein for each of the plurality of proteins, removing data from the full multiple sequence alignment for the protein comprises:
removing one or more amino acid sequences from the multiple sequence alignment for the protein.

3. The method of claim 2, wherein removing one or more amino acid sequences from the multiple sequence alignment for the protein comprises:
sampling a reduction parameter value from a set of possible reduction parameter values in accordance with a probability distribution over the set of possible reduction parameter values, wherein the reduction parameter value specifies a number of amino acid sequences to be removed from the full multiple sequence alignment for the protein; and
removing the specified number of amino acid sequences from the full multiple sequence alignment for the protein.

4. The method of claim 3, wherein removing the specified number of amino acid sequences from the full multiple sequence alignment for the protein comprises:
randomly selecting the amino acid sequences to be removed from the full multiple sequence alignment for the protein.

5. The method of claim 1, wherein for each of the plurality of proteins, masking data from the full multiple sequence alignment for the protein comprises:
masking an identity of a respective amino acid at one or more positions in one or more amino acid sequences in the full multiple sequence alignment for the protein.

6. The method of claim 5, wherein masking an identity of a respective amino acid at one or more positions in one or more amino acid sequences in the full multiple sequence alignment for the protein comprises:
randomly sampling the positions to be masked in the amino acid sequences in the full multiple sequence alignment for the protein.

7. The method of claim 5, further comprising training the structure prediction neural network to, for each of the plurality of proteins, process the representation of the reduced multiple sequence alignment for the protein to generate an auxiliary output that predicts the identity of each masked amino acid in the reduced multiple sequence alignment for the protein.

8. The method of claim 1, wherein determining the target structure parameters for the protein based on the output structure parameters for the protein comprises:
adding random noise values to the output structure parameters for the protein.

9. The method of claim 1, wherein the structure prediction neural network is configured to process a network input that comprises both: (i) a representation of a multiple sequence alignment for a protein, and (ii) a representation of an amino acid sequence of the protein.

10. The method of claim 1, further comprising, for each of the plurality of proteins, determining a confidence estimate for the target structure parameters for the protein.

11. The method of claim 10, further comprising:
identifying one or more proteins for which the confidence estimate for the target structure parameters for the protein does not satisfy a threshold; and
refraining from training the structure prediction neural network on the identified proteins.

12. The method of claim 10, wherein training the structure prediction neural network comprises:
determining gradients of an objective function that measures, for one or more of the plurality of proteins, an error between: (i) the structure parameters generated by the structure prediction neural network by processing the representation of the reduced multiple sequence alignment for the protein, and (ii) the target structure parameters for the protein, wherein the error is scaled by a function of the confidence estimate for the target structure parameters for the protein.

13. The method of claim 10, wherein for each of the plurality of proteins:
the confidence estimate for the target structure parameters for the protein is generated as an auxiliary output of the structure prediction neural network by processing the representation of the full multiple sequence alignment of the protein;
wherein the confidence estimate for the target structure parameters for the protein defines an estimate of an error between: (i) the output structure parameters generated by the structure prediction neural network by processing the full multiple sequence alignment of the protein, and (ii) ground truth structure parameters characterizing a ground truth structure of the protein.

14. The method of claim 1, further comprising training the structure prediction neural network to, for one or more other proteins, process a representation of a multiple sequence alignment for the other protein to generate structure parameters that match ground truth structure parameters for the other protein.

15. The method of claim 14, wherein the ground truth structure parameters for the other proteins are determined by physical experiments.

16.-24. (canceled)

25. The method of claim 1, in which wherein the structure parameters comprise one or both of a plurality of torsion angles and a plurality of atom coordinates.

26. The method of claim 1, further including obtaining an amino acid sequence of a protein and using the trained structure prediction neural network to determine a structure of the protein.

27. The method of claim 26 further including extracting the protein from a human or animal body and obtaining the amino acid sequence from the extracted protein.

28.-33. (canceled)

34. A system comprising:
one or more computers; and
one or more storage devices communicatively coupled to the one or more computers, wherein the one or more storage devices store instructions that, when executed by the one or more computers, cause the one or more computers to perform operations for training a structure prediction neural network that is configured to generate structure parameters that characterize a structure of a protein by processing a network input that comprises a representation of a multiple sequence alignment for the protein, the method comprising:
obtaining, for each of a plurality of proteins, a full multiple sequence alignment for the protein;
generating, for each of the plurality of proteins, target structure parameters characterizing a structure of the protein from the full multiple sequence alignment for the protein, comprising:
processing a representation of the full multiple sequence alignment for the protein using the structure prediction neural network to generate output structure parameters characterizing a structure of the protein; and
determining the target structure parameters for the protein based on the output structure parameters for the protein;
determining, for each of the plurality of proteins, a reduced multiple sequence alignment for the protein, comprising removing or masking data from the full multiple sequence alignment for the protein; and
training the structure prediction neural network to, for one or more of the plurality of proteins, process a representation of the reduced multiple sequence alignment for the protein to generate structure parameters that match the target structure parameters for the protein.

35. (canceled)

36. One or more non-transitory computer storage media storing instructions that when executed by one or more computers cause the one or more computers to perform operations for training a structure prediction neural network that is configured to generate structure parameters that characterize a structure of a protein by processing a network input that comprises a representation of a multiple sequence alignment for the protein, the method comprising:
obtaining, for each of a plurality of proteins, a full multiple sequence alignment for the protein;
generating, for each of the plurality of proteins, target structure parameters characterizing a structure of the protein from the full multiple sequence alignment for the protein, comprising:
processing a representation of the full multiple sequence alignment for the protein using the structure prediction neural network to generate output structure parameters characterizing a structure of the protein; and
determining the target structure parameters for the protein based on the output structure parameters for the protein;
determining, for each of the plurality of proteins, a reduced multiple sequence alignment for the protein, comprising removing or masking data from the full multiple sequence alignment for the protein; and
training the structure prediction neural network to, for one or more of the plurality of proteins, process a representation of the reduced multiple sequence alignment for the protein to generate structure parameters that match the target structure parameters for the protein.
